(19) United States
(12) Patent Application Publication
(10) Pub. No.: US 2004/0068005 A1
(43) Pub. Date: Apr. 8, 2004

(54) Pharmaceutical combinations

(76) Inventors: Zoltan Szilvassy, Debrecen (HU); Arpad Tosaki, Debrecen (HU); Jozsef Nemeth, Pecs (HU); Peter Kovacs, Debrecen (HU); Csaba Pankucsi, Debrecen (HU); Ferenc Hernadi, Debrecen (HU); Peter Ferdinandy, Szeged (HU)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/332,946

(22) PCT Filed: Jul. 13, 2001
(86) PCT No.: PCT/HU01/00079

(30) Foreign Application Priority Data
Jul. 14, 2000 (HU) P 0002628

(51) Int. Cl. A61K 31/21; A61K 38/28

(52) U.S. Cl. 514/509; 514/3

(57) ABSTRACT

Pharmaceutical combinations for treatment and/or prevention of all sorts, periods and complications of diabetes mellitus in mammals, thus including the pre-diabetic diseases and their complications, optionally including furthermore ischaemic heart disease comprising an effective dose of at least one enzymatic nitric oxide (NO) donor active ingredient and optionally comprising an effective dose of at least one antidiabetic active ingredient, and further optionally comprising usual pharmaceutically acceptable carriers and/or other auxiliaries. The combination may consist of more than one pharmaceutical compositions. The effective doses related to the new insulin-sensitizing effect are considerably lower than the usual doses related to the know effect of most active substances dues to metabolic effects that influence insulin sensitivity in healthy and insulin resistant mammals. The usual dose of NO-donors is necessary when the patient has also ischaemic heart disease. Preferred antidiabetics include insulin, a thiazolidinedion, a biguanide derivative, an α-glucosidase-inhibitor, and α2-adrenergic-antagonist and/or a sulphonamide, preferably a sulphonylurea. Preferred enzymatic NO donors are nitroglycerin, racemic isosorbide monoitrate, and/or its stereoisomers, racemic isosorbide dinitrate and/or its stereoisomers, erythityl tetranitrate, pentaerythritol-tetranitrate, methylpropyl-propanediol-dinitrate, propatyl nitrate, trolnitrate, tenitramine and/or nicorandile. The invention includes methods of treatment and processes to prepare the compositions.

The object of this invention is a pharmaceutical combination for treatment and prevention of diabetes mellitus, including all sorts, periods and complications of diabetes mellitus, thus including the pre-diabetic diseases and their complications, including furthermore diabetic ischaemic heart disease associated with diabetes mellitus, comprising optionally more than one pharmaceutical composition, whereby at least one of the compositions comprises an effective dose of at least one enzymatic nitric oxide (NO) donor and optionally an effective dose of at least one antidiabetic active ingredient and optionally the usual pharmaceutically acceptable carriers and/or other auxiliaries.

The basis of the invention is the recognition of a new insulin-sensitizing effect and synergism using enzymatic NO donors for monotherapy or in combined therapy with a conventional antidiabetic active ingredient, mainly with a per os antidiabetic active ingredient. It was found that besides the known vascular effect enzymatic NO donors have a metabolic (hypoglycaemic/antihyperglycaemic) effect as well. Thus the present invention represents a fundamentally new antidiabetic therapeutic approach using an antianginal agent with metabolic effect.

Where not otherwise stated the following definitions and abbreviations are used further on:

Diabetes: all sorts, periods and complications of diabetes mellitus, including all diseases associated with diabetes mellitus, and pre-diabetic diseases and their complications.

Main complications of diabetes:

diseases based on diabetic microvascular problems, including but not limited to diabetic neuropathy, retinopathy, nephropathy; diabetes associated ischaemic heart disease, including myocardial ischaemic heart disease,

disturbances in gastric and intestinal motility, such as gastroparesis arid problems of sphincter of ODDI.

Insulin-dependent (Type I.) diabetes mellitus: IDDM

Non-insulin dependent (Type II.)diabetes mellitus: NIDDM

Polycistic ovary syndrome (diabetic disease): PCOS

Gestational diabetes syndrome (pre-diabetic disease): GDM

nitroglycerin (enzymatic NO donor): NTG

racemic isosorbide mononitrate, and/or its stereoizomers: (enzymatic NO donor): ISMN

racemic isosorbide dinitrate and/or its stereoizomers: (enzymatic NO donor): ISDN

3-morpholinosydnonimine (non-enzymatic NO donor): SIN-1

It is known that IDDM results from a decreased insulin production by the pancreatic β-cells. NIDDM is known as a heterogeneous disease resulting from a dynamic interaction between defects in insulin secretion and insulin action.

Mechanisms responsible for the β-cell failure are not totally clarified, but may be related to the chronic demands placed on the β-cells by peripherial insulin resistance and/or the effect of hyperglycemia to impair β-cell function. The β-cell failure may also occur as an independent inherent defect in “pre-diabetic” individuals. NIDDM often develops from certain at risk populations, such as indviduals with polycistic ovary syndrome which is the most common endocrine disorder in women of reproductive age.

NO donor drugs are widely used in ischaemic heart disease and for the treatment of cardiac failure. Such medications can be used in a variety of formulations with different routes of administration: parenteral (intravenous, intramuscular, subcutaneous) transdermal (patch, oinment), rectal or enteral (sublingual, buccal, per os in liquid or solid forms).

NO donors have two basic groups: non-enzymatic NO donors, and enzymatic NO donors. Non-enzymatic NO donors release NO spontaneuosly by chemical degradation, while enzymatic NO donors require an enzymatic process.

Non-enzymatic NO donors include sydnonimine-derivatives (SIN-1), sodium nitropusside, S-nitroso-N-acetyl-D,L-penicillamine, sodium nitrite.

Enzymatic NO donors include NTG, ISMN, ISDN, erythrityl tetranitrate, pentaerythritol tetranitrate, methyl-propyl-propanediol-dinitrate, propatylnitrate, trolnitrate, tenitramine, nicorandile. Nitroglycerin is a prototype of the enzymatic NO donors.

Recently, the non-enzymatic NO donor SIN-1 has been reported to inhibit insulin release in isolated pancreatic islets (Am. J. Physiol 1996;271;C1098-C1102). Insulin sensitivity was further reported to be increased through stimulation of NO production in the liver (patent application No PCT/US/99/23098) using non-enzymatic NO donors such as SIN-1, sodium nitrite, sodium nitropusside, and S-nitroso-N-acetyl-D,L-penicillamine.

Using enzymatic NO donors is a basic recognition of our invention, while non-enzymatic NO donors, such as SIN-1 are not suitable for treatment of diabetes mellitus. This is summarized as follows:

It is known that enzymatic NO donors do not release NO in coronary vessels with a diameter smaller than 100 μm. Thus although dilating supepicardial arteries including stenotic segments and collateral vessels in the coronary vasculature they do not dilate coronary microvessels i.e. resistance coronary vessels. This selective effect renders enzymatic NO donors ‘safe coronary vasodilators’ due to the minimum or no risk of the ‘coronary steal’ phenomenon characteristic for other pure vasodilators such as slow Ca²⁺ channel blockers or phosphodiesterase inhibitors. Non-enzymatic NO donors however (due to spontaneous NO release) are known to dilate coronary conductance (>100 μm) and resistance (<100 μm) vessels, as well.

Beyond vascular effect, enzymatic NO donors are known to elicit several favourable biological actions, such as inhibition of platelet aggregation, inhibition Ca²⁺ entry into cardiac myocytes, inhibition of catecholamine release from cardiac adrenergic nerve terminals, and other actions in the central nervous system and immune system as well. Non-enzymatic NO-donors have several unfavourable effects as summarized below:

According to recent publications, NO produced by non-enzymatic NO donors play an important role in the destruction of the pancreatic β-cells thereby leading to IDDM. Therefore, any therapeutic use of the non-enzymatic NO donors to enhance insulin-sensitivity in NIDDM is clinically contraindicated. It means, that the said compounds worsen diabetes mellitus. Furthermore, the mortality of the so-called ‘deadly quartet’ (NIDDM, obesity, dislypidemia, and hypertension) is mainly (80%) due to ischemic heart disease and/or cardiac failure. During chemical degradation SIN-1 is known to produce super-oxide anion (O₂⁻) and NO, and the chemical reaction of these two radicals gives peroxinitrite (ONOO⁻), the toxic properties of which are well known (Exp. Toxicol Pathol 1999;51:517-21).

Also cytotoxic effects of non-enzymatic NO donors were reported.

In a clinical investigation (Metabolism 2000;49:313-318), the hypothesis was tested that sodium nitroprusside would increase insulin-mediated glucose uptake in humans. In the control group insulin was infused using the euglycemic clamp protocol. It was found that systemic infusion of sodium nitroprusside did not increase insulin-mediated glucose disposal neither in young nor in old subjects.

It is known that NIDDM can be treated initially using monotherapy with known oral antidiabetic agents (such as sulphonylureas, biguahides, α-glucosidase inhibitors, benzoic acid derivatives, thiazolidinediones, α2-receptor antagonists) but will eventually require the combination of said compounds, and in most patients, an additional insulin therapy will be needed. Long-term control of blood glucose levels in IDDM and NIDDM will decrease the incidence and prolong the time until progression but will not inhibit complications such as diabetic retinopathy, nephropathy, and neuropathy. (J. Natl.Med. Assoc. 1991, 91, 389-395; Ann Intern Med. 1999, 131, 281-303).

The recognition of our invention thus includes:

a.) monotherapy with an enzymatic NO donor results in a hypoglycaemic/antihyperglycaemic effect in humans and other mammals, and

b.) the combination of an enzymatic NO donor, with an antidiabetic active ingredient, particularly a per os agent, results in an increase of the hypoglycaemic/antihyperglycaemic effect of the antidiabetic active ingredient while providing protection against myocardial ischaemia, a commonly occurring complication of NIDDM, and further that fect is reduced due to the insulin sensitizing effect of the enzymatic NO donor.

As already indicated above an object of our invention is a pharmaceutical combination for treatment and prevention of diabetes mellitus, including all sorts, periods and complications of diabetes mellitus, thus including the prediabetic diseases and their complications, including furthermore diabetic ischaemic heart disease associated with diabetes mellitus, comprising optionally more than one pharmaceutical compositions, whereby at least one of the compositions comprises an effective dose of at least one enzymatic NO donor active ingredient and optionally comprises an effective dose of at least one antidiabetic active ingredient, and further optionally comprises usual pharmaceutically acceptable carriers and/or other auxiliaries.

Further object of the invention is a pharmaceutical combination comprising at least one composition comprising an effective dose of at least one enzymatic NO donor and at least one composition comprising an effective dose of an antidiabetic active ingredient and any of the compositions optionally comprising usual pharmaceutically acceptable carriers and/or other auxiliaries.

Another object of the invention is a pharmaceutical composition for treatment and prevention of diabetes mellitus, including all sorts, periods and complications of diabetes mellitus, thus including the pre-diabetic diseases and their complications, including furthermore diabetic ischaemic heart disease associated with diabetes mellitus, comprising an effective dose of at least one enzymatic NO donor active ingredient and optionally comprising an effective dose of at least one antidiabetic active ingredient, and further optionally comprising usual pharmaceutically acceptable carriers and/or other auxiliaries.

It is evident from the above the combinations according to the invention include three embodiments of the invention: a formulation (composition) containing a NO-donor, a formulation containing both a NO-donor and an antidiabetic together and more than one formulation (composition) where the NO-donor and the antidiabetic appear in separated formulations.

Another aspect of our invention is a combination or composition for treatment and prevention of diabetes associated complications, preferably of diabetic microvascular problems, such as diabetic neuropathy, retinopathy, nephropathy, and of diabetes associated ischaemic heart disease, particularly myocardial ischaemic heart disease, of disturbances in gastric and intestinal motility, particularly of gastroparesis, and problems of sphincter of ODDI, and of pre-diabetic diseases, such as polycistic ovary syndrome (PCOS), and of gestational diabetes syndrome (GDNM)

The combination or composition may comprise an organic nitrate compound as enzymatic NO donor, and insulin or a per os antidiabetic active ingredient, preferably thiazolidinedion, biguanide derivative, α-glucosidase-inhibitor, α2-adrenergic-antagonist and/or a sulphonamide, preferably a sulphonylurea as the antidiabetic active ingredient.

According to the preferred embodiment the enzymatic NO donor is nitroglycerin (NTG), racemic isosorbide mononitrate, and/or its stereoizomers (ISIS), racemic isosorbide dinitrate and/or its stereoizomers (ISDN), erythrityl tetranitrate, pentaerytritol-tetranitrate, methylpropyl-propanediol-dinitrate, propatyl nitrate, trolnitrate, tenitramine and/or nicorandile, and the antidiabetic active ingredient is insulin, troglitazone, pioglitazone, rosiglitazone, meglitinide analogues, acetohexamide, carbutamide, chlorpropamide, glibenclamide, glibornuride, glibutamide, gliclazide, glipizide, glimepiride, gliquidone, glisentide, glisolamide, glisoxepide, glybuzole, glyclopyramide, glycyclamide, glymidine free acid and its salts, metahexamide, tolazamide, tolbutamide, metformine, phenformine, buformine, idazoxane, acarbose, miglitol, and/or voglibose.

An important embodiment of the invention is a combination or composition comprising as the enzymatic NO donor nitroglycerin, racemic isosorbide mononitrate and/or its stereoizomers, racemic isosorbide dinitrate and/or its stereoizomers, and as the antidiabetic active ingredient insulin, troglitazone, pioglitazone, rosiglitazone, glibenclamide, metformine and/or idazoxane.

The compositions according to the invention may be formulated for direct medical use for parenteral (intravenous, intramuscular, subcutaneous), transdermal (patch or oinment), per os liquid and solid (tablet, spray, liquid), rectal, nasal, sublingual, buccal administration for controlled (sustained) or usual release.

Another important aspect of our invention is, that we have found, that the effective doses related to the new insulin-sensitizing effect are considerably lower than the usual doses related to the known effect of most active substances (see data of the following preferred embodiments) Our results have shown for the first time that in addition to favourable effects on the heart and vasculature, nitro-glycerin, isosorbide-5-mononitrate, and all of the other enzymatic NO donors at a dose lower than used for the treatment of stable angina pectoris (see Table 1) produces metabolic effects that influence insulin sensitivity in healthy and insulin resistant patients and mammals.

The higher dose is only necessary, when the patient has also to be treated against the “classical” disease, the ischaemic heart disease.

Preferred embodiments of our invention are formulations comprising the following daily or per hour effective doses for NO-treatment alone:

| | | |
|---|---|---|
| NTG sustained-release, p.os. | 0.5-31.2 | mg |
| NTG transdermal oinment | 0.2-0.8 | mg/hour |
| NTG transdermal patch | 0.2-0.8 | mg/hour |
| ISDN tablet, capsule | 0.3-135 | mg |
| ISDN sustained-release, p.os. | 0.2-160 | mg |
| ISDN transdermal | 3-180 | mg |
| ISMN tablet, capsule | 1-40 | mg |
| ISMN sustained-release, p.os | 2-240 | mg |
| ISMN transdermal | 40-300 | mg |

A preferred combination or composition comprises the following combinations of two active ingredients using the following daily or per hour effective doses

a.) As Enzymatic NO Donor:

| | | |
|---|---|---|
| NTG retard per os | 0.26-31.2 | mg |
| NTG transdermal | 0.02-0.8 | mg/hour |
| NTG transd. oinment | 0.02-0.8 | mg/hour |
| ISDN per os | 0.1-135 | mg |
| ISDN retard per os | 0.2-160 | mg |
| ISDN transdermal | 3-180 | mg |
| ISMN per os | 0.1-120 | mg |
| ISMN retard per os | 0.2-240 | mg |
| ISMN transdermal | 3-300 | mg |

b.) As Per Os Antidiabetic Active Ingredient:

| | | |
|---|---|---|
| glibenclamide, per os | 0.75-14 | mg |
| metformin, per os | 50-3000 | mg |
| glyburide | 0.1-100 | mg |
| idazoxan, per os | 20-600 | mg |
| troglitazon | 20-600 | mg |
| pioglitazon | 5-50 | mg |
| rosiglitazon | 5-50 | mg |
| insulin, i.v. | 4-500 | NE/ml. |

For the purpose of our invention more than two active ingredients are applicable. The following variations are preferred combinations using effective doses as exemplified above:

enzymatic NO donor, sulphonylurea, and/or biguanide derivative; or

enzymatic NO donor, sulphonylurea, and/or thiazolidinedione derivative; or

enzymatic NO donor, sulphonylurea derivative and/or insulin; or

enzymatic NO donor, biguanide, and/or tiadiazolidindion derivative; or

enzymatic NO donor, biguanide derivative and/or insulin; or

enzymatic NO donor, thiazolidinedione derivative and/or insulin; or

enzymatic NO donor, sulphonylurea, biguanide, and/or thiazolidinedione derivative; or

enzymatic NO donor, sulphonylurea, biguanide, thiazolidinedione derivative and/or insulin.

The present invention is also directed to a process for the preparation of combinations or compositions of the invention by way of formulating an effective dose for direct medical use of active substances using the usual pharmaceutically acceptable carriers and/or other auxiliaries for pharmaceutically acceptable application.

Another object of our invention is a method of treatment and prevention of diabetes mellitus, including all sorts, periods and complications of diabetes mellitus, thus including the pre-diabetic diseases and their complications, including furthermore diabetic ischaemic heart disease associated with diabetes mellitus, by way of administering to the patient in need of such treatment an effective dose of a pharmaceutical combination comprising optionally more than one pharmaceutical composition, whereby at least one of the compositions comprises an effective dose of at least one enzymatic nitric oxide donor active ingredient and optionally comprises an effective dose of at least one antidiabetic active ingredient, and further optionally comprises usual pharmaceutically acceptable carriers and/or other auxiliaries.

The pharmaceutical combination used for the treatment may comprise at least one composition comprising an effective dose of at least one enzymatic NO donor and at least one composition comprising an effective dose of an antidiabetic active ingredient and any of the compositions optionally comprising usual pharmaceutically acceptable carriers and/or other auxiliaries.

The treatment may be carried out using a pharmaceutical composition comprising an effective dose of at least one enzymatic NO donor active ingredient. The composition may optionally comprise an effective dose of at least one antidiabetic active ingredient, and further optionally comprising usual pharmaceutically acceptable carriers and/or other auxiliaries.

Another object of our invention is a method of treatment and prevention of diabetes associated complications, particularly of diabetic microvascular problems, such as diabetic neuropathy, retinopathy, nephropathy, and of diabetes associated ischaemic heart diseases, such as myocardial ischaemic heart disease as well as of disturbances in gastric and intestinal motility, preferably of gastroparesis, and problems of sphincter of ODDI, and further of pre-diabetic diseases, preferably polycistic ovary syndrome (PCOS), and of gestational diabetes syndrome (GDM) by way of administering to the patient in need of such treatment an effective dose of the combinations and compositions according to the invention.

According to preferred embodiments the treatments are accomplished by way of administering an organic nitrate compound as enzymatic NO donor, and insulin, a per os antidiabetic active ingredient, preferably thiazolidinedion, biguanide derivative, α-glucosidase-inhibitor, α2-adrenergic-antagonist and/or a sulphonamide, preferably a sulphonylurea, as the antidiabetic active ingredient.

A preferred method includes administering to the patient in need of such treatment an effective dose of a pharmaceutical combination or composition comprising as the enzymatic NO donor nitroglycerin (NTG), racemic isosorbide mononitrate, and/or its stereoizomers (ISMN), racemic isosorbide dinitrate and/or its stereoizomers (ISDN), erythrityl tetranitrate, pentaerytritol-tetranitrate, methylpropyl-propanediol-dinitrate, propatyl nitrate, trolnitrate, tenitramine and/or nicorandile, and as the antidiabetic active ingredient insulin, troglitazone, pioglitazone, rosiglitazone, meglitinide analogues, acetohexamide, carbutamide, chlorpropamide, glibenclamide, glibornuride, glibutamide, gliclazide, glipizide, glimepiride, gliquidone, glisentide, glisolamide, glisoxepide, glybuzole, glyclopyramide, glycyclamide, glymidine free acid and its salts, metahexamide, tolazamide, tolbutamide, metformine, phenformine, buformine, idazoxane, acarbose, miglitol, and/or voglibose.

A preferred method consists in administering an effective dose of as the enzymatic NO donor nitroglycerin, racemic isosorbide mononitrate and/or its stereoizomers, racemic isosorbide dinitrate and/or its stereoizomers and as the antidiabetic active ingredient insulin, troglitazone, pioglitazone, rosiglitazone, glibenclamide, metformine, idazoxane.

More particularly administering an effective dose of as the enzymatic NO donor nitroglycerin, and of as the antidiabetic active ingredient troglitazone, pioglitazone, rosiglitazone, or metformine is preferable.

Another object of our invention is a method of monotherapic treatment by way of administering a composition according to following effective daily or per hour doses:

| | | |
|---|---|---|
| NTG sustained-release, p.os. | 0.5-31.2 | mg |
| NTG transdermal oinment | 0.2-0.8 | mg/hour |
| NTG transdermal patch | 0.2-0.8 | mg/hour |
| ISDN tablet, capsule | 0.3-135 | mg |
| ISDN sustained-release, p.os. | 0.2-160 | mg |
| ISDN transdermal | 3-180 | mg |
| ISMN tablet, capsule | 1-40 | mg |
| ISMN sustained-release, p.os | 2-240 | mg |
| ISMN transdermal | 40-300 | mg |

Further object of our invention is a method of combined therapy treatment by way of administering a combination and/or composition according to following effective daily or per hour doses using two active substances

a.) As Enzymatic NO Donor:

| | | |
|---|---|---|
| NTG retard per os | 0.26-31.2 | mg |
| NTG transdermal | 0.02-0.8 | mg/hour |
| NTG transd. oinment | 0.02-0.8 | mg/hour |
| ISDN per os | 0.1-135 | mg |
| ISDN retard per os | 0.2-160 | mg |
| ISDN transdermal | 3-180 | mg |
| ISMN per os | 0.1-120 | mg |
| ISMN retard per os | 0.2-240 | mg |
| ISMN transdermal | 3-300 | mg |

b.) As Per Os Antidiabetic Active Ingredient

| | | |
|---|---|---|
| glibenclamide, per os | 0.75-14 | mg |
| metformin, per os | 50-3000 | mg |
| glyburide | 0.1-100 | mg |
| idazoxan, per os | 20-600 | mg |
| troglitazon | 20-600 | mg |
| pioglitazon | 5-50 | mg |
| rosiglitazon | 5-50 | mg |
| insulin, i.v. | 4-500 | NE/ml |

Another object of our invention is a method of combined therapy treatment by way of administering a combination and/or composition according to effective daily or per hour doses as above, using variations of more than two active substances:

enzymatic NO donor, sulphonylurea, and/or biguanide derivative; or

enzymatic NO donor, sulphonylurea, and/or thiazolidinedione derivative; or

enzymatic NO donor, sulphonylurea derivative and/or insulin; or

enzymatic NO donor, biguanide, and/or tiadiazolidindion derivative; or

enzymatic NO donor, biguanide derivative and/or insulin; or

enzymatic NO donor, thiazolidinedione derivative and/or insulin; or

enzymatic NO donor, sulphonylurea, biguanide, and/or thiazolidinedione derivative; or

enzymatic NO donor, sulphonylurea, biguanide, thiazolidinedione derivative and/or insulin.

In the following we summerize some of the main benefits of our invention:

Nitroglycerin a prototype of enzymatic NO donors enhance insulin sensitivity in humans, and thus combination of sulphonylureas with an enzymatic NO donor yielded an amalgamation of benefit in controlling NIDDM metabolic disorder epecially in patients at risk of ischeamic heart disease.

Another aspect of the benefit from a sulphonylurea-enzymatic NO donor combination derives from the prevalence of gastrointestinal motility disorders in diabetes. These alterations are considered to result from the other major complication of diabetes i.e. peripheral neuropathy. As a result, disturbances occur in gastric and intestinal motility (diabetic gastroparesis) as well as in gall-bladder and sphincter of Oddi motility. The latter is of crucial importance in the development of gall-stone disease in diabetes. Moreover, K_{ATP} activation is an important mechanism in nitrergic relaxation of the sphincter of Oddi, thus, sulphonylurea derivatives may at least in part block physiological sphincter of Oddi relaxation function even in the absence of diabetes. Therefore when a sulphonylurea derivative is combined with as enzymatic NO donor, beyond producing a synergistic effect on glucose metabolism, the combination bears preservation of gastrointestinal sphincter function with special regards to the sphincter of Oddi. Finally, as the antidiabetic dose of a sulphonylurea in the presence of enzymatic NO donors is lower than that used in monotherapy, the risk of hypoglycaemia will also be lower.

Metformin one of the biuanides treat obese NIDDM patients. However, besides inducing lactate acidosis of low incidence, this drug was contraindicated in cardiac and respiratory insufficiency. Due to the insulin sensitizing effect of enzymatic NO donors, a potentiating synergism between enzymatic NO donors and metformin on blood glucose lowering effect allow the antidiabetic dose of metformin to decrease, moreover, the vascular effects and direct myocardial protection induced by anzymatic NO donors would make metformin therapy safer in NIDDM patients at risk of myocardial disease.

The major disadvantage of α-glucosidase inhibitors was gastrointestinal intolerance due to both osmotic effects and bacterial fermentation of undigested carbohydrates. These effects, however, exhibited dose dependence.

Since enzymatic NO donors further reduce insulin release with a potentiating effect on the hypoglyceamic performance of insulin, an enzymatic NO donor—acerbose combination would yield a reduction in the antihyperglyceamic dose of acarbose, therefore reducing its gastrointestinal side effects.

The major disadvantage of the use of thiazolidinediones derives from their dose-dependent toxic effects produced aneamia and liver damage. Combining thiazolidinediones with enzymatic NO donors reduced the antidiabetic dose of thiazolidinediones resulting in a decrease in their potential to produce toxic effects. Moreover, the protective effect of the two drugs against myocardial ischaemia further decreases the incidence of ischaemic heart disease in NIDDM patients.

In diabetic state, the insulin sensitivity enhancing effi-cacy of enzymatic NO donors overcome their inhibitory effect on insulin release in favour of a hypoglycaemic action at modest insulin release. In addition, enzymatic NO donors counteract the hypertensive effect of α₂-receptor antagonists besides conferring protection on the ischaemic heart.

Since enzymatic NO donors enhance insulin sensitivity, the combination of insulin with enzymatic NO donors necessitates lower and less frequently applied insulin doses when insulin is the therapeutic possibility. Thus, risk of the two most important complications of chronic insulin therapy such as body weight gain and insulin resistance: is significantly de—

### EXPLANATION OF THE FIGURES

FIG. 1. Effect of nitroglycerin patch (0.4 mg/hour) on oral glucose tolerance test in healthy volunteers. The data are means±SD, * placebo vs. active patch at p<0.05

[symbol] Placebo patch, insulin, n=20

[symbol] Active patch, insulin, n=20

[symbol] Placebo patch, glucose, n=20

[symbol] Active patch, glucose, n=20

FIG. 2/A Interaction between different treatments on insulin sensitivity in normal conscious rabbits. The data are means±SD.

Control-1, n=6; Control-2, n=7; Nitroglycerin patch 0.07 mg/kg/h; Troglitazon 70 mg/kg p.os, n=6; Metformin 5 mg/kg i.v., n=6; Glibenclamide 1 mg/kg i.v., n=6; Idazoxan 2 mg/kg i.v., n=6

*: significant vs. control-1, p<0.05

#: significant vs. control-2, p<0.05

FIG. 2/B Interaction between different treatments on insulin sensitivity in hypercholesterolaemic, insulin resistant conscious rabbits. The data are means±SD

Control-1, n=6; Control-2, n=6; Nitroglycerin patch 0.07 mg/kg/h; Troglitazon 70 mg/kg p.os, n=6; Metformin 5 mg/kg i.v., n=6; Glibenclamide 1 mg/kg i.v., n=6; Idazoxan 2 mg/kg i.v., n=6

+: significant vs. placebo patch, p<0.05

FIG. 3 Interaction between ISMN and metformin on insulin sensitivity in normal and hypercholesterolaemic, insulin resistant-conscious rabbits. The data are means±SD

Placebo-1, n=6; Placebo-2, n=6; Metformin 100 mg/kg p.os, n=6

ISMN: isosorbide-5-mononitrate 5 mg/kg p.os n=6;

HC-IR: hypercholesterolaemic, insulin resistant

*: significant vs. placebo-1, p<0.05

#: significant vs. placebo-2, p<0.05

FIG. 4/A Synergism between enzymatic NO donors and insulin sensitizers in hyrpercholesterolaemic, insulin resistant conscious rabbits. The data are expressed as percent of means.

HC-IR: hypercholesterolaemic, insulin resistant

NTG: nitroglycerin; ISMN: isosorbide-5-mononitrate

*: significant vs. HC-IR control, p<0.05

FIG. 4/B Synergism between nitroglycerin and non insulin sensitizig antihyperglycaemic compounds in hypercholesterolaemic, insulin resistant conscious rabbits. The data are expressed as percent of means.

HC-IR control, n=6; HC-IR: hypercholesterolaemic, insulin resistant; Nitroglycerin patch 0.07 mg/kg/h; Glibenclamide 1 mg/kg i.v., n=6; Idazoxan 2 mg/kg i.v., n=6; Combinations, n=6

*: significant vs. HC-IR control, p<0.05

FIG. 5/A Effect different treatments on nerve conduction velocity in femoral “C” fibers in streptozotocin diabetic rabbits. The data are means±SD.

Control-1, n=6; Control-2, n=6; Nitroglycerin patch 0.07 mg/kg/h; Troglitazon 70 mg/kg p.os, n=6; Metformin 5 mg/kg i.v., n=6; Glibenclamide 1 mg/kg i.v., n=6; Idazoxan 2 mg/kg 1.v., n=6

*: significant vs. control-1, p<0.05

FIG. 5/B Effect of nitroglycerin and nitroglycerintroglitazon combination on nerve conduction velocity in femoral “C” fibers in hypercholesterolaemic, insulin resistant rabbits. The data are means±SD.

Control-1, n=6; Control-2, n=6; Nitroglycerin patch 0.07 mg/kg/h; Troglitazon 70 mg/kg p.os, n=6

*: significant vs. control-1, p<0.05

#: significant vs. control-2, p<0.05

+: significant vs. placebo patch, p<0.05

FIG. 6/A Interaction between different treatments on ventricular pacing induced ischaemia in normal conscious rabbits. The data are means±SD

Control-1, n=6

Control-2, n=6; Nitroglycerin patch 0.07 mg/kg/h; Troglitazon 70 mg/kg p.os, n=6; Metformin 5 mg/kg i.v., n=6; Glibenclamide 1 mg/kg i.v., n=6; Idazoxan 2 mg/kg i.v., n=6

*: significant vs. control-1, p<0.05

+: significant vs. placebo patch, p<0.05

FIG. 6/B Interaction between different treatments on ventricular pacing-induced ischaemia in hypercholesterolaemic, insulin resistant rabbits. The data are means±SD

Control-1, n=6; Control-2, n=6; Nitroglycerin patch 0.07 mg/kg/h; Troglitazon 70 mg/kg p.os, n=6; Metformin 5 mg/kg i.v., n=6; Glibenclamide 1 mg/kg i.v., n=6; Idazoxan 2 mg/kg i.v., n=6

*: significant vs. control-1, p<0.05

#: significant vs. control-2, p<0.05

FIG. 7 Treshold doses of per os controlled-release isosorbide-5-mononitrate (ISMN) on insulin sensitivity and myocardial ischaemia in normal conscious rabbits. The data are means±SD. * placebo vs. treatment on insulin sensitivity and # placebo vs. treatment on ST-elevation, p<0.05.

[symbol] Glucose infusion rate (insulin sensitivity)

[symbol] ST-elevation (ventricular pacing-induced ischaemia)

The following examples illustrate various aspects of the invention without the aim of limitation.

### EXAMPLES

### Method 1

### Effect of Nitroglycerin on Glucose-Stimulated Insulin Release in Humans

Twenty persons were studied. None of them had a history of hypertension, diabetes mellitus, or smoking.

Between 2 oral glucose, tolerance tests (OGTT) the volunteers were randomized for receiving either active NITRODERM TTS 10 (releasing approx. 0.4 mg hours⁻¹ nitroglycerin) or placebo patches. The study was carried out and evaluated in a double blind fashion. The patients received the glucose solution at 8 a.m. Venous blood samples were taken during fasting (immediately before glucose) and at 15, 30, 60, 90, 120 and 180 minutes after the glucose load. The samples evaluated for plasma glucose level (mmol 1⁻¹) and immunoreactive insulin responses (\check{e}U ml⁻¹). Plasma glucose was determined by means of an autoanalyzer, using the glucoseoxidease method. Immunoreactive insulin levels were assessed by radioimmunoassay method using antihuman antibody. During the study, two ECG leads (II and V₁ or V₆) and arterial blood pressure were continuously monitored.

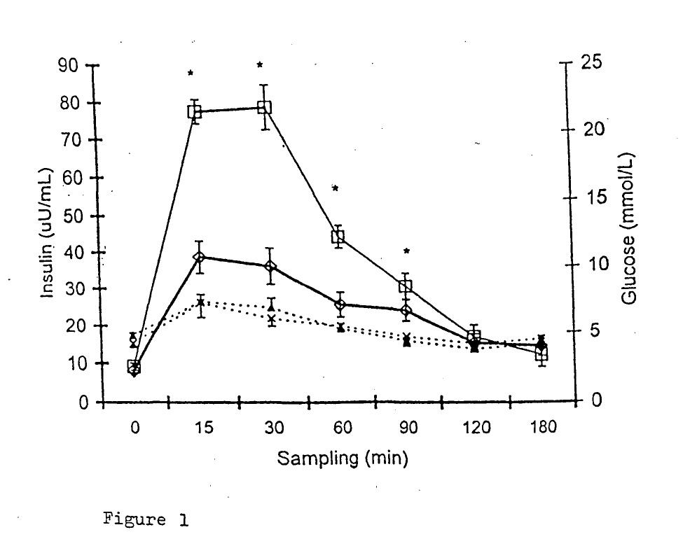

Nitroglycerin significantly decreased the increase in insulin in response tooral glucose load as compared to corresponding values in the placebo patch group. There were no differences in plasma glucose levels in the two groups (FIG. 1 and Table I).

### Method 2

### Animals and Surgery

The experiments are carried out with adult, male New Zealand white rabbits, weighing 3.0-3.5 kg, housed as described in Am. J. Physiol 1994, Heart Circ. Physiol, 35: H2033-H2041 and J. Moll Cell.. Cardiol. 1997, 29: 1977-1983.

### Cardiac Electrophysiology and Haemodynamics

The right intracavitary electrogram, the chest-lead ECG, the left ventricular pressure curve and MABP are continuously recorded as above.

### Global Myocardial Ischaemia Induced by Ventricular Overdrive Pacing

The induction of global myocardial ischaemia was based on a method described as above.

### Experimental Hyperlipidaemia and Insulin Resistance in Rabbits

Adult male New Zealand white rabbits are fed laboratory chow enriched with 1.5% cholesterol (atherosclerotic group) over a period of eight to twelve weeks. According to our experiences, exposure to cholesterol-enriched diet over this period results in a nearly twentyfold increase in serum total cholesterol level, a decrease in insulin-stimulated glucose uptake with aortic lesion surface area of 55% and an approx. fifty percent loss of endothelium-dependent relaxation of rings from thoracic aortae in rabbits.

### Nerve Conduction Velocity Studies

These series of experiments were carried out to verify/exclude sensory neuropathy. Left saphenous nerve conduction velocity was determined as described in Eur J. Pharmacol 1999, 386:83-88.

### Determination of Insulin Sensitivity/Insulin Resistance

To estimate insulin sensitivity and/or insulin resistance, hyperinsulinaemic (100 \check{e}U/ml) euglycaemic (5.5 mM/l) glucose clamping was used. The glucose clamping values (M) were established as values of glucose infusion rates expressed in mg/kg/min to maintain blood glucose level at 5.5 mM/l.

### Results

### 1. Interaction Between Nitroglycerin and Glibenclamide, Metformin, Troglitazon or Idazoxan on Insulin Sensitivity in Normal Conscious Rabbits

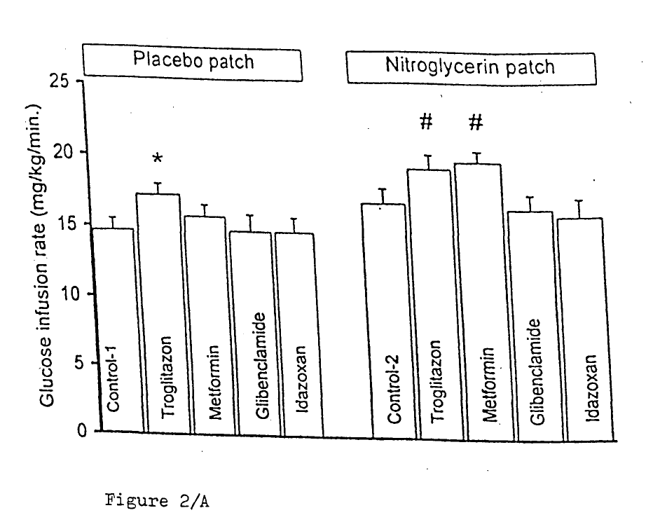

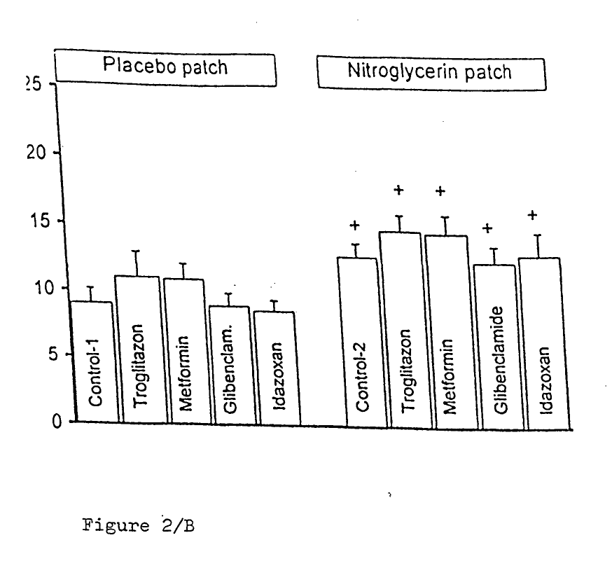

As shown in FIG. 2/A and Table II, the M values of hyperinsulinaemic and euglycaemic glucose clamping significantly increased in the presence of troglitazon (70 mg/kg/day p. os over 3 days). Either metformin (5 mg/kg/day i.v. over 3 days) or glibenclamide (1 mg/kg i.v. 10 min prior to glucose infusion) or idazoxan. (2 mg/kg i.v.) was without effect.

The M values are also significantly increased in the presence of 0.07 mg/kg/min transdermal nitroglycerin combined with troglitazon or metformin (FIG. 2/A, Table II).

### 2. Interaction Between Nitroglycerin and Glibenclamide, Metformin, Troglitazon or Idazoxan on Insulin Sensitivity in Hypercholesterolaemic, Insulin Resistant Conscious Rabbits

In the presence of nitroglycerin (0, 0.7 mg/kg/h) M values of hrperinsulinaemic and euglycaemic glucose clamping increased as shown in FIG. 2/B (control-2, Table II). In the presence of nitroglycerin and troglitazon (70 mg/kg/day p.os over 3 days), metformin (5 mg/kg/day i.v. over 3 days), glibenclamide (1 mg/kg i.v. 10 min prior to glucose infusion) or idazoxan (2 mg/kg i.v.) the M values are also significantly increased compared to the placebo patch group (FIG. 2/B, Table II).

### 3. Interaction Between Isosorbide-5-mononitrate and Metformin, on Insulin Sensitivity in Normal Conscious Rabbits

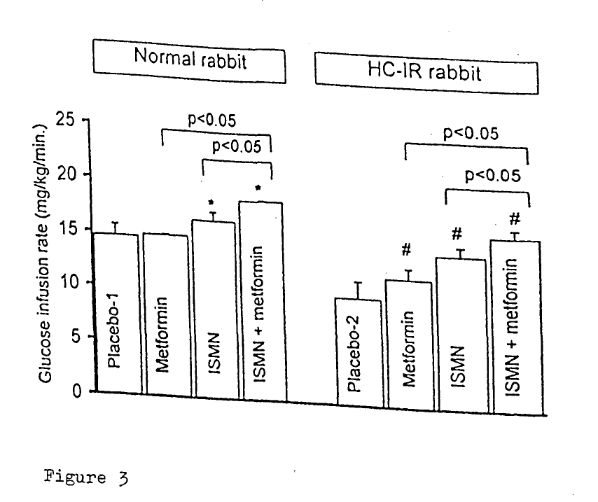

After treatment with isosorbide-5-mononitrate (ISIAN) (1 mg/kg/day, Olicard 40, Solvay Pharma, Hannover, Germany) M values of hyperinsulinaemic and euglycaemic glucose clamping increased as compared to the control (FIG. 3, placebo-1; Table III) value. Metformin (100 mg/kg/day p.os over 3 days) was without any effect, but in the presence of ISML, metformin elicited a significant increase in the M values (FIG. 3, Table III).

### 4. Interaction Between Isosorbide-5-mononitrate and Metformin, on Insulin Sensitivity in Hypercholesterolaemic, Insulin Resistant Conscious Rabbits

After treatment with isosorbide-5-mononitrate (ISMN) (5 mg/kg/day, Olicard 40, Solvay Pharma, Hannover, Germany) M values of hyperinsulinaemic and euglycaemic glucose clamping significantly increased as compared to the control (FIG. 3, placebo-2) value. Metformin (100 mg/kg/day p.os over 3 days) alone and the combination of ISMN and metformin also elicited a significant increase in the M values (FIG. 3, Table III). 1.

### 5. Synergism Between Enzymatic NO Donors and Insulin Sensitizer Compounds on Insulin Sensitivity in Hypercholesterolaemic, Insulin Resistant Conscious Rabbits

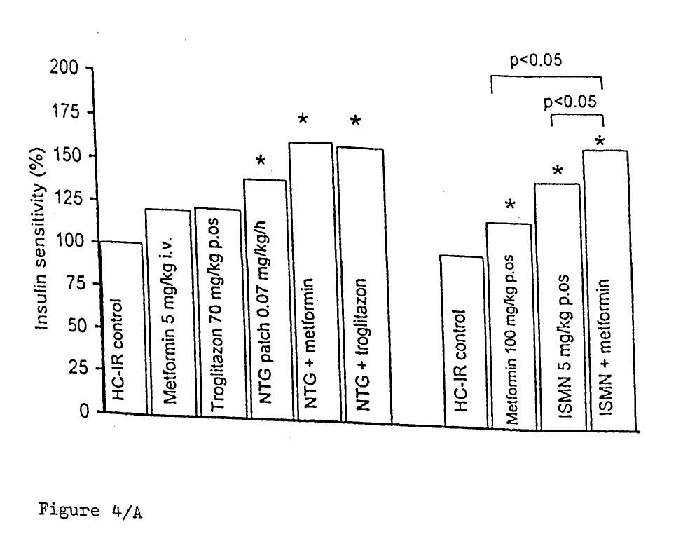

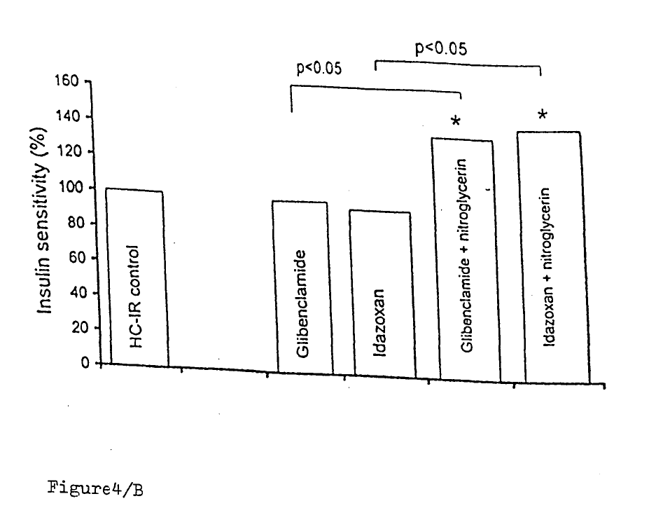

The enzymatic NO donors (nitroglycerin, ISMN) combined with known insulin sensitizer compounds (metformin, troglitazon) produced synergism (increased effectivity) on insulin sensitivity, as shown in FIG. 4/B and Table IV.

### 6. Synergism Between Enzymatic NO Donors and Non Insulin Sensitising Antihyperglycaemic Compounds in Hypercholesterolaemic, Insulin Resistant Conscious Rabbits

Nitroglycerin combined with antihyperglycaemic compounds that are without insulin sensitising effect (glibenclamide, idazoxan) produced synergism (increased effectivity) on glycaemic control (FIG. 4/B, Table IV).

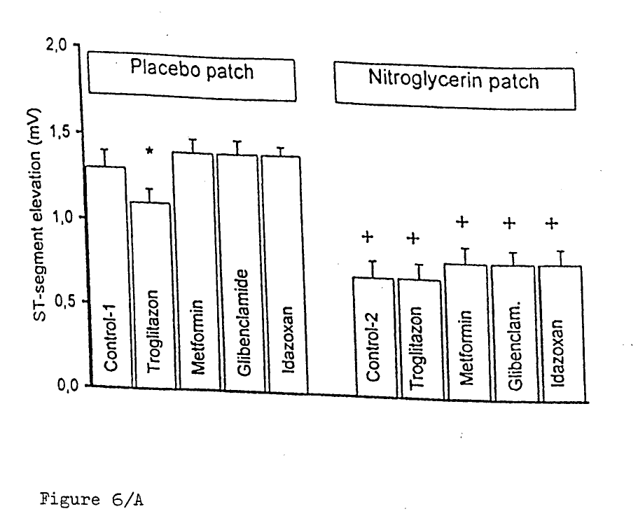

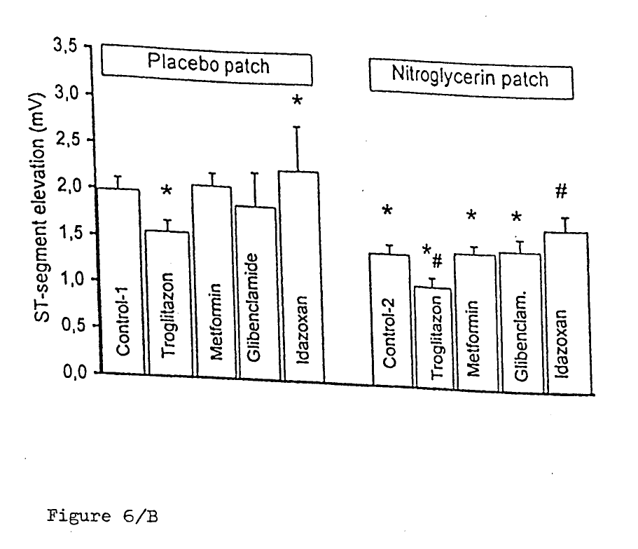

Nitroglycerin and glibeclamide combination produced synergism not only on metabolic effects, but on anti-ischaemic effects (FIG. 6/B, Table VI), as well.

### 7. Interaction Between Nitroglycerin and Glibenclamide, Metformin, Troglitazon or Idazoxan on Femoral Nerve Conduction Velocity in Anaesthetized Rabbits with Streptozotocin Diabetes

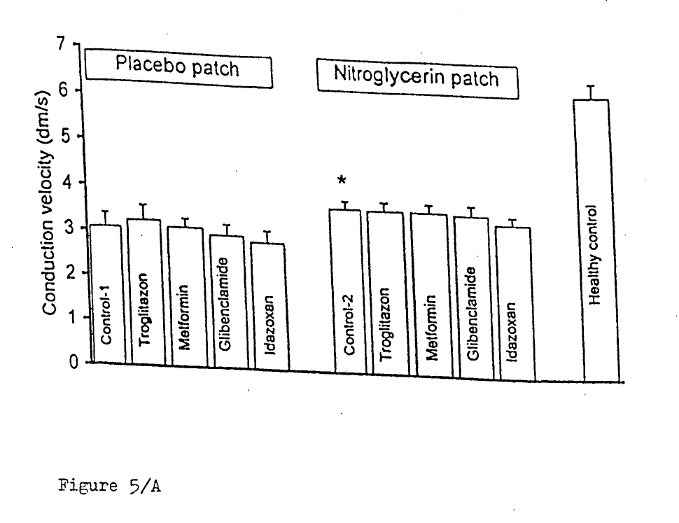

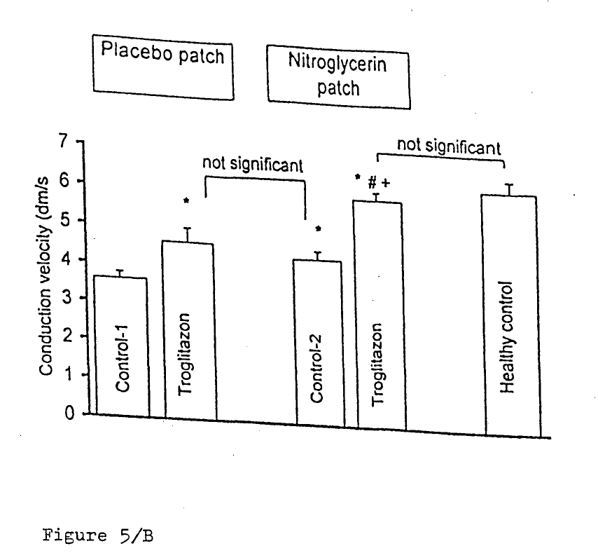

Streptozotocin (40 mg/kg i.v.) produced a decrease in nerve conduction velocity in femoral “A” and “C” fibres, respectively, as determined 8 weeks after streptozotocin injection. Nitroglycerin (12-h patch on vs 12-h patch off periods over three days) significantly improved nerve conduction in “C” fibres (FIG. 5/A, Table V) with a marginal amelioration of “A” fibre conduction (data not shown). Neither troglitazon,nor glibenclamide, idazoxan or metformin produced any effect on nerve conduction velocity in the streptozotocin-diabetes model. The combination of any of these drugs with nitroglycerin yielded an improvement of conduction velocity in streptozotocin-diabetic aminals similar to that seen with nitroglycerin alone.

### 8. Synergism Between Enzymatic NO Donors and Insulin Sensitizer Compounds on Femoral Nerve Conduction Velocity in Anaesthetized Rabbits with Hyperlipidaemia-Induced Insulin Resistance

An eight-week period of atherogenic diet resulted in a decrease in nerve conduction velocity in “C” fibres (FIG. 5/B.; control-1; Table V). This was attenuated by nitroglycerin and troglitazon (FIG. 5/B, control-2; Table V).

When these drugs were applied together, the “C” fibre nerve conduction velocity values increased further, and did not differ significantly from those measured in healthy rabbits in the absence of any drugs (FIG. 5/B, Table V).

### 9. Interaction Between Nitroglycerin and Glibenclamide, Metformin, Troglitazon or Idazoxan on Pacing-Induced Myocardial Ischaemia in Normal Conscious Rabbits

Troglitazon and nitroglycerin significantly decreased intra cavitary ST-segment elevation (FIG. 6/A, Table VI), but met formin and glibenclamide were without effect.

When nitroglycerin was combined with troglitazon, glibencla mide, metformin or idazoxan, each combination produced an anti-ischaemic effect similar to that produced by nitroglyc erin alone (FIG. 6/A, Table VI).

### 10. Interaction Between Nitroglycerin and Glibenclamide, Metformin, Troglitazon or Idazoxan on Pacing-Induced Myocardial Ischaemia in Hypercholesterolaemic, Insulin Resistant Conscious Rabbits

Nitroglycerin significantly decreased intracavitary ST-segment elevation (FIG. 6/B; control-2; Table VI.). Troglitazon also induced an anti-ischaemic effect although of lower amplitude than that produced by nitroglycerin. Metformin and glibenclamide were without effect. Idazoxan aggravated ischaemic changes produced by VOP (FIG. 6/B, Table VI), moreover, VOP induced ventricular extrasystolesin the insulin resistant animals.

When nitroglycerin was combined with troglitazon the anti-ischaemic effect exceeded that induced by nitroglycerin alone (FIG. 6/B, Table VI). Nitroglycerin+metformin, nitroglycerin+glibenclamide produced an anti-ischaemic effect approx. of the same magnitude as that seen with nitroglycerin alone.

### 11. Threshold Doses of per os Extended-Release Isosorbide-5-mononitrate on Insulin Sensitivity and Ventricular Pacing-Induced Myocardial Ischaemia in Normal Conscious Rabbits

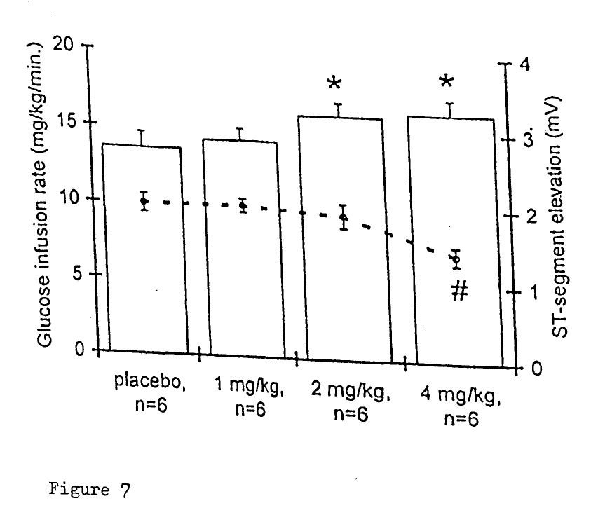

The effect of 1, 2, and 4 mg/kg per os ISMN were tested, to compare the metabolic (insulin sensitising) and anti-ischaemic (changes in ST-segment elevation) dose range of ISMN. 1 mg/kg was without any effect, but 2 mg/kg produced significant metabolic effect with a statistically insignificant anti-ischaemic effect. 4 mg/kg and higher doses of ISMN had metabolic and anti-ischaemic effects, as well (FIG. 7, Table VII). The threshold dose of the insulin sensitising effect was 2 mg/kg, 50% of the threshold dose of the anti-ishaemic effect.

The usual human anti-anginal (anti-ischaemic) doses of controlled release ISMN preparations are 30 to 240 mg/day. The threshold anti-anginal dose is 30 mg/day.

Human dose: 15 mg retard ISMN+250 mg retard metfromin

### Evaluation of Experimental Results

Our results have shown for the first time that in addition to favourable effects on the heart and vasculature, nitro-glycerin, ISMN, and all of the other enzymatic NO donors at a dose identical or even lower to that used for the treatment of stable angina pectoris (see Table 1) produces metabolic effects, that influence insulin sensitivity in healthy and insulin resistant patients and mammals.

The enzymatic NO-donors applied at an anti-ischaemic dosage range (see the corresp. Table) produces additional metabolic effects, that influence post-prandial haemodynamic adjustments of potential risk in patients and mammals with coronary artery disease.

The combinations of the known antihyperglycaemic compounds with enzymatic NO donors produce synergism on insulin sensitivity.

TABLE 1
Doses of the Enzymatic NO-Donors Used for the
Treatment of Stable Angina Pectoris

| | drug | form | mg | daily administration |
|---|---|---|---|---|
| 1. | NTG | sublingual tablet | 0.3-0.6 | 1-3 times or 2-5 min. |
| | | | | before activity |
| 2. | NTG | sublingual spray | 0.4-0.8 | 1-3 times or 2-5 min. |
| | | | | before activity |
| 3. | NTG | buccal tablet | 1-3 | 3 times or 2-5 min. before |
| | | | | activity |
| 4. | NTG | sustained release, | 2.6-10.4 | 2-3 times |
| | | oral | | |
| 5. | NTG | transdermal | 1-10 | 3-4 times |
| | | | cm | |
| | | ointment, 2% | 7.5-30 | |
| | | | mg | |
| 6. | NTG | transdermal patch | 0.2-0.8 | once |
| | | | mg/hour | |
| 7. | ISDN | oral spray | 1.25-3.75 | 1-3 times or 2-5 min. |
| | | | | before activity |
| 8. | ISDN | sublingual tablet | 2.5-10 | 5-10 min. before activity |
| 9. | ISDN | tablet, capsule | 10-45 | 3 times |
| 10. | ISDN | sustained release, | 20-80 | 1-2 times |
| | | oral | | |
| 11. | ISDN | transdermal | 30-90 | 1-2 times |
| 12. | ISMN | tablet, capsule | 10-20 | twice |
| 13. | ISMN | sustained release, | 30-240 | once |
| | | oral | | |

TABLE 2
Effect of transdermal nitroglycerin on oral glucose tolerance test
(OGTT) in healthy volunteers.

| | Placebo patch | | Active patch | |
|---|---|---|---|---|
| | Glucose, | Insulin, | Glucose, | Insulin, |
| Sampling | mmol l⁻¹ | μU ml⁻¹ | mmol l⁻¹ | μU ml⁻¹ |
| 0 (control) | 4.8 ± 0.2 | 9.1 ± 0.9 | 4.2 ± 0.3 | 8.2 ± 0.9 |
| 15 min | 7.4 ± 0.5⁺ | 77.7 ± 3.3⁺ | 7.3 ± 1.1⁺ | 38.9 ± 4.4^{#+} |
| 30 min | 6.9 ± 0.7⁺ | 78.8 ± 6.1⁺ | 6.1 ± 0.6⁺ | 36.3 ± 5.0^{#+} |
| 60 min | 5.3 ± 0.4⁺ | 44.3 ± 3.1⁺ | 5.5 ± 0.4 | 25.6 ± 3.3^{#+} |
| 90 min | 4.4 ± 0.2 | 30.6 ± 3.7⁺ | 4.7 ± 0.7 | 24.1 ± 2.9^{#+} |
| 120 min | 3.8 ± 0.4 | 16.8 ± 3.3⁺ | 4.2 ± 0.3 | 15.2 ± 2.4⁺ |
| 180 min | 4.1 ± 0.4 | 12.1 ± 3.1 | 4.5 ± 0.5 | 14.4 ± 2.6 |

The data are means ± S.D. obtained with 20 patients.
⁺different from ‘0’ values at p < 0.05; ^{#}placebo vs active patch at p < 0.05.

TABLE I
Effect of transdermal nitroglycerin on oral glucose tolerance test
in human volunteers

| | |
|---|---|
| Placebo patch | Active patch |

| Sampling | Glucose | Insulin | Glucose | Insulin |
|---|---|---|---|---|
| (min) | (mmoL⁻¹) | (μU mL⁻¹) | (mmoL⁻¹) | (μU mL⁻¹) |
| 0 | 4.8 ± 0.2 | 9.1 ± 0.9 | 4.2 ± 0.3 | 8.2 ± 0.9 |
| (control) | | | | |
| 15 | 7.4 ± 0.5 | 77.7 ± 3.3 | 7.3 ± 1.1 | 38.9 ± 4.4 |
| 30 | 6.9 ± 0.7 | 78.8 ± 6.1 | 6.1 ± 0.6 | 36.3 ± 5.0 |
| 60 | 5.3 ± 0.4 | 44.3 ± 3.1 | 5.5 ± 0.4 | 25.6 ± 3.3 |
| 90 | 4.4 ± 0.2 | 30.6 ± 3.7 | 4.7 ± 0.7 | 24.1 ± 2.9 |
| 120 | 3.8 ± 0.4 | 16.8 ± 3.3 | 4.2 ± 0.3 | 15.2 ± 2.4 |
| 180 | 4.1 ± 0.4 | 12.1 ± 3.1 | 4.5 ± 0.5 | 14.4 ± 2.6 |

The data are mean ± SD obtained from 20 patients.

TABLE II
Interaction between different treatments on insulin sensitivity in rabbits

| | Glucose infusion rate (mg/kg/min.) | | | |
|---|---|---|---|---|
| | | | Hypercholesterolaemic, | |
| | Normal rabbit | | insulin resistant rabbit | |
| | | Active patch | | Active patch |
| | Placebo | (nitroglycerin | Placebo | (nitroglycerin |
| | patch | 0.07 mg/kg/h) | patch | 0.07 mg/kg/h) |
| Control | 14.667 ± | 16.857 ± | 9.000 ± | 12.667 ± |
| | 0.8165 | 1.0690 | 1.0954 | 1.0328 |
| | n = 6 | n = 7 | n = 6 | n = 6 |
| Troglitazon, | 17.167 ± | 19.333 ± | 11.000 ± | 14.667 ± |
| n = 6 | 0.7528 | 1.0328 | 1.8974 | 1.2111 |
| (70 mg/kg | | | | |
| p.os) | | | | |
| Metformin, | 15.667 ± | 19.833 ± | 10.833 ± | 14.500 ± |
| n = 6 | 0.8165 | 0.7528 | 1.1690 | 1.3784 |
| (5 mg/kg i.v.) | | | | |
| Glibenclamide, | 14.667 ± | 16.500 ± | 8.833 ± | 12.333 ± |
| n = 6 | 1.2111 | 1.0488 | 0.9832 | 1.2111 |
| (1 mg/kg i.v.) | | | | |
| Idazoxan, | 14.667 ± | 16.000 ± | 8.500 ± | 12.833 ± |
| n = 6 | 1.0328 | 1.2649 | 0.8367 | 1.7224 |
| (2 mg/kg i.v.) | | | | |

The data are mean ± SD.

TABLE III
Interaction between ISMN and metformin on insulin sensitivity
in rabbits

| | Glucose infusion rate (mg/kg/min.) | |
|---|---|---|
| | | Hypercholesterolaemic, |
| Treatment | Normal rabbit | insulin resistant rabbit |
| Control, n = 6 | 14.60 ± 1.0300 | 9.70 ± 1.6000 |
| Metformin, n = 6 | 14.80 ± 0.0800 | 11.70 ± 1.0300 |
| (100 mg/kg p.os) | | |
| ISMN controlled release, | | |
| n = 6 | 16.30 ± 0.8200 | 14.00 ± 0.8900 |
| (5 mg/kg p.os) | | |
| ISMN controlled release (5 | | |
| mg/kg p.os) + metformin | 19.30 ± 0.820 | 15.90 ± 0.7500 |
| (100 mg/kg p.os), n = 6 | | |

The data are mean ± SD.

TABLE IV
Synergism between enzymatic NO donors and known anti-diabetics in
hypercholesterolaemic, insulin resistant rabbits

| | Insulin sensitivity |
|---|---|
| Treatment | (%) |
| Control | 100.0 |
| Metformin (5 mg/kg i.v.) | 120.4 |
| Troglitazon (70 mg/kg p.os) | 122.2 |
| Nitroglycerin patch (0.07 mg/kg/h) | 140.7 |
| Nitroglycerin patch (0.07 mg/kg/h) + metformin | 163.0 |
| (5 mg/kg i.v.) | |
| Nitroglycerin patch (0.07 mg/kg/h) + troglitazon | 161.1 |
| (70 mg/kg p.os) | |
| Metformin (100 mg/kg p.os) | 120.6 |
| ISMN controlled release (5 mg/kg p.os) | 144.4 |
| ISMN controlled release (5 mg/kg p.os) + Metformin | 163.9 |
| (100 mg/kg p.os) | |

TABLE V
Interaction between different treatments on nerve
conduction velocity in femoral
“C” fibers in rabbits

| | Nerve conduction velocity (dm/s)) |
|---|---|
| Healthy untreated | 6.2 ± 0.3 |
| rabbit | |

| | Streptozotocin | | Hypercholesterolaemic, | |
|---|---|---|---|---|
| | diabetic rabbit | | insulin resistant rabbit | |
| | Placebo | Active | Placebo | Active |
| Treatment | patch | patch | patch | patch |
| Control, n = 6 | 3.1 ± 0.3 | 3.6 ± 0.2 | 3.6 ± 0.2 | 4.3 ± 0.4 |
| Troglitazon, n = 6 | 3.2 ± 0.3 | 3.6 ± 0.2 | — | — |
| (70 mg/kg p.os) | | | | |
| Metformin, n = 6 | 3.1 ± 0.2 | 3.6 ± 0.2 | — | — |
| (5 mg/kg i.v.) | | | | |
| Glibenclamide, n = 6 | 2.9 ± 0.24 | 3.6 ± 0.2 | — | — |
| (1 mg/kg i.v.) | | | | |
| Idazoxan, n = 6) | 2.8 ± 0.26 | 3.4 ± 0.2 | — | — |
| (2 mg/kg i.v.) | | | | |
| Troglitazon, n = 6 | — | — | 4.6 ± 0.4 | 5.9 ± 0.2 |
| (70 mg/kg p.os) | | | | |

The data are mean ± SD.

TABLE VI
Interaction between different treatments on ventricular pacing-induced
ischaemia in rabbits

| | ST-segment elevation (mV) | | | |
|---|---|---|---|---|
| | | | Hypercholesterolaemic, | |
| | Normal rabbit | | insulin resistant rabbit | |
| | | Active patch | | Active patch |
| | Placebo | (nitroglycerin | Placebo | (nitroglycerin |
| Treatment | patch | 0.07 mg/kg/h) | patch | 0.07 mg/kg/h) |
| Control, n = 6 | 1.3 ± 0.10 | 0.7 ± 0.10 | 2.0 ± 0.13 | 1.4 ± 0.10 |
| Troglitazon, | 1.1 ± 0.08 | 0.7 ± 0.09 | 1.6 ± 0.12 | 1.08 ± 0.10 |
| n = 6 | | | | |
| (70 mg/kg | | | | |
| p.os) | | | | |
| Metformin, | 1.4 ± 0.08 | 0.8 ± 0.10 | 2.1 ± 0.13 | 1.5 ± 0.08 |
| n = 6 | | | | |
| (5 mg/kg i.v.) | | | | |
| Glibenclamide, | 1.4 ± 0.08 | 0.8 ± 0.08 | 1.9 ± 0.40 | 1.5 ± 0.13 |
| n = 6 | | | | |
| (1 mg/kg i.v.) | | | | |
| Idazoxan, n = 6 | 1.4 ± 0.05 | 0.8 ± 0.09 | 2.3 ± 0.50 | 1.8 ± 0.16 |
| (2 mg/kg i.v.) | | | | |

The data are mean ± SD.

TABLE VII
Treshold doses of per os controlled release ISMN on insulin sensitivity
and myocardial ischaemia in normal rabbits

| | Glucose infusion rate | ST-segment elevation |
|---|---|---|
| Treatment | (mg/kg/min.) | (mV) |
| Control, n = 6 | 13.6 ± 1.03 | 2.0 ± 0.12 |
| 1 mg/kg ISMN, n = 6 | 14.2 ± 0.8 | 2.0 ± 0.09 |
| 2 mg/kg ISMN, n = 6 | 16.0 ± 0.9 | 1.9 ± 0.16 |
| 4 mg/kg ISMN, n = 6 | 16.3 ± 1.0 | 1.4 ± 0.12 |

The data are mean ± SD.

1. An insulin-sensitizing pharmaceutical composition or combination comprising
a) as an active ingredient an insulin-sensitizing effective dose of an enzymatic nitric oxide (NO) donor organic nitrate
b) optionally in combination with an antidiabetic effective dose of insulin or at least one per os antidiabetic as further active ingredient
c) and further optionally comprising pharmaceutically acceptable carriers and/or other auxiliaries
for prevention and treatment of all sorts, periods and complications of diabetes mellitus.

2. The combination according to claim 1 for prevention and treatment of pre-diabetic diseases and their complications, and furthermore diabetic ischaemic heart disease associated with diabetes mellitus.

3. A pharmaceutical combination according to claim 1, comprising at least one composition comprising an effective dose of at least one enzymatic NO donor and at least one composition comprising an effective dose of an antidiabetic active ingredient and any of the compositions optionally comprising usual pharmaceutically acceptable carriers and/or other auxiliaries.

4. A pharmaceutical composition according to claim 1 for treatment and prevention of diseases according to claim 1, comprising an effective dose of at least one enzymatic nitric oxide (NO) donor active ingredient and optionally comprising an effective dose of at least one antidiabetic active ingredient, and further optionally comprising usual pharmaceutically acceptable carriers and/or other auxiliaries.

5. A pharmaceutical composition according to claim 1 comprising an insulin-sensitizing effective dose of an enzymatic nitric oxide (NO) donor organic nitrate as active ingredient and further comprising usual pharmaceutically acceptable carriers and/or other auxiliaries.

6. A composition or combination according to any of claims 1 to 5 for insulin-sensitizing and treatment of diabetes associated ischaemic heart disease comprising in addition to the insulin-sensitivizing effective dose a further amount of NO-donor to include the anti-anginal effective dose.

7. A combination or composition according to any of claims 1 to 6 comprising an organic nitrate compound as enzymatic NO donor, and insulin, a per os antidiabetic active ingredient, preferably thiazolidinedion, biguanide derivative, α-glucosidase-inhibitor, α2-adrenergic-antagonist and/or a sulphonamide, preferably a sulphonylurea, as the antidiabetic active ingredient.

8. A combination or composition according to any of claims 1 to 7 comprising as the enzymatic NO donor nitroglycerin (NTG), racemic isosorbide mononitrate, and/or its stereoizomers (ISMN), racemic isosorbide dinitrate and/or its stereoizomers (ISDN), erythrityl tetranitrate, pentaerytritol-tetranitrate, methylpropyl-propanediol-dinitrate, propatyl nitrate, trolnitrate, tenitramine and/or nicorandile, and as the antidiabetic active ingredient insulin, troglitazone, pioglitazone, rosiglitazone, meglitinide analogues, acetohexamide, carbutamide, chlorpropamide, glibenclamide, glibornuride, glibutamide, gliclazide, glipizide, glimepiride, gliquidone, glisentide, glisolamide, glisoxepide, glybuzole, glyclopyramide, glycyclamide, glymidine free acid and its salts, metahexamide, tolazamide, tolbutamide, metformine, phenformine, buformine, idazoxane, acarbose, miglitol, and/or voglibose.

9. A combination or composition according to any of claims 1 to 6 comprising as the enzymatic NO donor nitroglycerin, racemic isosorbide mononitrate and/or its stereoizomers, racemic isosorbide dinitrate and/or its stereoizomers, as the antidiabetic active ingredient insulin, troglitazone, pioglitazone, rosiglitazone, glibenclamide, metformine, idazoxane.

10. A combination or composition according to any of claims 1 to 3 for treatment and prevention of diabetes associated complications, preferably of diabetic microvascular problems, preferably diabetic neuropathy, retinopathy, nephropathy, and of diabetes associated ischaemic heart disease, preferably myocardial ischaemic heart disease, of disturbances in gastric and intestinal motility, preferably of gastroparesis, and problems of sphincter of ODDI, and of pre-diabetic diseases, preferably polycistic ovary syndrome (PCOS), and of gestational diabetes syndrome (GDM)

11. A combination or composition according to any of claims 1 to 7 formulated for parenteral (intravenous, intramuscular, subcutaneous), transdermal (patch), per os liquid and solid (tablet, spray, liquid), nasal, sublingual, buccal administration for controlled (sustained) and usual release.

12. A composition according to any of claims 1 to 13 comprising the following daily or per hour effective doses for insulin-sensitizing:

| | | |
|---|---|---|
| NTG sustained-release p.os. | 0.5-31.2 | mg |
| NTG transdermal patch | 0.2-0.8 | mg/hour |
| ISDN tablet, capsule | 0.3-135 | mg |
| ISDN sustained-release, p.os. | 0.2-160 | mg |
| ISDN transdermal | 3-180 | mg |
| ISMN tablet, capsule | 1-40 | mg |
| ISMN sustained-release, p.os | 2-240 | mg |
| ISMN transdermal | 40-300 | mg |

13. A composition according to 8 comprising the following daily or per hour effective doses:

| | | |
|---|---|---|
| NTG sustained-release,p.os. | 5.2-31.2 | mg |
| NTG transdermal patch | 0.2-0.8 | mg/hour |
| ISDN tablet, capsule | 3-135 | mg |
| ISDN sustained-release p.os | 2-160 | mg |
| ISDN transdermal | 3-180 | mg |
| ISMN tablet, capsule | 20-40 | mg |
| ISMN sustained-release p.os | 30-240 | mg |
| ISMN transdermal | 40-300 | mg |

14. A composition according to any of claims 1 to 13 comprising the following daily or per hour effective doses for insulin-sensitizing:

| | | |
|---|---|---|
| NTG sustained-release, p.os. | 0.5-5.2 | mg |
| NTG transdermal patch | 0.2-0.8 | mg/hour |
| ISDN tablet, capsule | 0.3-3 | mg |
| ISDN sustained-release, p.os. | 0.2-2 | mg |
| ISDN transdermal | 3-180 | mg |
| ISMN tablet, capsule | 1-20 | mg |
| ISMN sustained-release, p.os. | 2-30 | mg |
| ISMN transdermal | 40-300 | mg |

15. A combination or composition according to claim 8 comprising the following daily or per hour effective doses for insulin-sensitizing and treatment of ischaemia:
a) as enzymatic NO donor:

| | | |
|---|---|---|
| NTG retard per os | 0.26-31.2 | mg |
| NTG transdermal | 0.02-0.8 | mg/hour |
| ISDN per os | 0.1-135 | mg |
| ISDN retard per os | 0.2-160 | mg |
| ISDN transdermal | 3-180 | mg |
| ISMN per os | 0.1-120 | mg |
| ISMN retard per os | 0.2-240 | mg |
| ISMN transdermal | 3-300 | mg |

b) as antidiabetic active ingredient:

| | | |
|---|---|---|
| glibenclamide per os | 0.75-14 | mg |
| metformin, per os | 50-3000 | mg |
| glyburide | 0.1-100 | mg |
| idazoxan per os | 20-600 | mg |
| troglitazon | 20-600 | mg |
| pioglitazon | 5-50 | mg |
| rosiglitazon | 5-50 | mg |
| insulin, i.v. | 4-500 | NE/ml. |

16. A combination or composition according to any of claims 1 and 8 comprising the following daily or per hour effective doses for insulin-sensitizing and treatment of ischaemia:
a) as enzymatic NO donor:

| | |
|---|---|
| NTG retard per os | 0.26-31.2 mg |
| NTG transdermal | 0.02-0.8 mg/hour |
| ISDN per os | 0.1-135 mg |
| ISDN retard per os | 0.2-160 mg |
| ISDN transdermal | 3-180 mg |
| ISMN per os | 0.1-120 mg |
| ISMN retard per os | 0.2-240 mg |
| ISMN transdermal | 3-300 mg |

b) as antidiabetic active ingredient:

| | |
|---|---|
| glibenclamide per os | 0.75-14 mg |
| metformin per os | 50-3000 mg |
| glyburide | 0.1-100 mg |
| idazoxan per os | 20-600 mg |
| troglitazon | 20-600 mg |
| pioglitazon | 5-50 mg |
| rosiglitazon | 5-50 mg |
| insulin i.v. | 4-500 NE/ml. |

17. A combination or composition according to any of claims 1 to 8 comprising the following daily or per hour effective doses for insulin sensitizing:
a) as enzymatic NO donor a substance of the group:

| | |
|---|---|
| NTG retard per os | 0.26-31.2 mg |
| NTG transdermal | 0.02-0.8 mg/hour |
| ISDN per os | 0.1-3 mg |
| ISDN retard per os | 0.2-2 mg |
| ISDN transdermal | 3-180 mg |
| ISDN per os | 0.1-20 mg |
| ISMN retard per os | 0.2-30 mg |
| ISMN transdermal | 3-300 mg |

b) as antidiabetic active ingredient a substance of the group:

| | |
|---|---|
| glibenclamide, per os | 0.75-14 mg |
| metformin per os | 50-3000 mg |
| glyburide | 0.1-100 mg |
| idazoxan per os | 20-600 mg |
| troglitazon | 20-600 mg |
| pioglitazon | 5-50 mg |
| rosiglitazon | 5-50 mg |
| insulin i.v. | 4-500 NE/ml |

18. A combination or composition according to any of claims 1 to 19 comprising the following combinations of more than two active ingredients using effective doses according to the claims 1 to 19:
enzymatic NO donor, sulphonylurea, and biguanide derivative; or
enzymatic NO donor, sulphonylurea, and thiazolidinedione derivative; or
enzymatic NO donor, sulphonylurea derivative and insulin; or
enzymatic NO donor, biguanide, and thiadiazolidindione derivative; or
enzymatic NO donor, biguanide derivative and insulin; or
enzymatic NO donor, thiazolidinedione derivative and/or insulin; or
enzymatic NO donor, sulphonylurea, biguanide, and thiazolidinedione derivative; or
enzymatic NO donor, sulphonylurea, biguanide, thiazolidinedione derivative and insulin.

19. Process for the preparation of a combination according to any of claims 1 to 15 characterised by formulating an effective dose for direct medical use of active substances using the usual pharmaceutically acceptable carriers and/or other auxiliaries for pharmaceutically acceptable application.

20. Method of treatment and prevention of diabetes mellitus, including all sorts, periods and complications of diabetes mellitus, thus including the pre-diabetic diseases and their complications, including further diabetic ischaemic heart disease associated with diabetes mellitus, characterised by administering to the patient in need of such treatment an effective dose of a combination or composition according to any of claims 1 to 19.

21. Method according to claim 20 for insulin-sensitizing and treatment of diabetes associated ischaemic heart disease comprising administering to a patient in need of such treatment in addition to the insulin-sensitivizing effective dose of enzymatic NO-donor organic nitrate a further amount of NO-donor to include the anti-anginal effective dose.

22. Method according Lo any of claims 20 to 21 characterised by administering an effective dose of nitroglycerin as the enzymatic NO donor, and of troglitazone, pioglitazone, rosiglitazone, or metformine as the antidiabetic active ingredient.

23. Method of monotherapic treatment according to any of claims 22 to 24 characterised by administering a composition according to following effective daily or per hour doses:

| | |
|---|---|
| NTG sustained-release, p.os. | 5.2-31.2 mg |
| NTG transdermal patch | 0.2-0.8 mg/hour |
| ISDN tablet, capsule | 3-135 mg |
| ISDN sustained-release, p.os. | 2-160 mg |
| ISDN transdermal | 3-180 mg |
| ISMN tablet, capsule | 20-40 mg |
| ISMN sustained-release, p.os | 30-240 mg |
| ISMN transdermal | 40-300 mg |

24. Method of monotherapic treatment according to any of claims 22 to 24 characterised by administering a composition according to following effective daily or per hour doses:

| | |
|---|---|
| NTG sustained-release, p.os. | 0.5-31.2 mg |
| NTG transderrnal patch | 0.2-0.8 mg/hour |
| ISDN tablet, capsule | 0.3-135 mg |
| ISDN sustained release, p.os. | 0.2-160 mg |
| ISDN transdermal | 3-180 mg |
| ISMN tablet, capsule | 1-40 mg |
| ISMN sustained-release. p.os | 2-240 mg |
| ISMN transdermal | 40-300 mg |

25. Method of monotherapic treatment according to any of claims 22 to 24 characterised by administering a composition according to following effective daily or per hour doses a member of the group:

| | |
|---|---|
| NTG sustained-release, p.os. | 0.5-5.2 mg |
| NTG transdermal patch | 0.2-0.8 mg/hour |
| ISDN tablet, capsule | 0.3-3 mg |
| ISDN sustained-release, p.os. | 0.2-2 mg |
| ISDN transdermal | 3-180 mg |
| ISMN tablet, capsule | 1-20 mg |
| ISMN sustained-release, p.os | 2-30 mg |
| ISMN transdermal | 40-300 mg |

26. Method of combined therapy treatment according to any of claims 22 to 24 characterised by administering a combination according to following effective daily or per hour doses using two active ingredients:
a) as enzymatic NO donor a member of the group:

| | |
|---|---|
| NTG retard per os | 0.26-31.2 mg |
| NTG transd. oinment | 0.02-0.8 mg/hour |
| ISDN per os | 1-135 mg |
| ISDN retard per os | 2-160 mg |
| ISDN transdermal | 3-180 mg |
| ISMN per os | 1-120 mg |
| ISMN retard per os | 3-240 mg |
| ISMN transdermal | 3-300 mg |

b) and as per os antidiabetic active ingredient a member of the group:

| | |
|---|---|
| glibenclamide per os | 0.75-14 mg |
| metformin per os | 50-3000 mg |
| glyburide | 0.1-100 mg |
| idazoxan per os | 20-600 mg |
| troglitazon | 20-600 mg |
| pioglitazon | 5-50 mg |
| rosiglitazon | 5-50 mg |
| insulin i.v. | 4-500 NE/ml. |

27. Method of combined therapy treatment according to any of claims 22 to 24 characterised by administering a combination according to following daily or per hour doses using two active ingredients:
a) as enzymatic NO donor of the group:

| | |
|---|---|
| NTG retard per os | 0.26-31.2 mg |
| NTG transd. oinment | 0.02-0.9 mg/hour |
| ISDN per os | 0.1-135 mg |
| ISDN retard per os | 0.2-160 mg |
| ISDN transdermal | 3-180 mg |
| ISMN per os | 0.1-120 mg |
| ISMN retard per os | 0.2-240 mg |
| ISMN transdermal | 3-300 mg |

b) and a per os antidiabetic of the group:

| | | |
|---|---|---|
| glibenclamide per os | 0.75-14 | mg |
| metformin, per os | 50-3000 | mg |
| glyburide | 0.1-100 | mg |
| idazoxan, per os | 20-600 | mg |
| troglitazon | 20-600 | mg |
| pioglitazon | 5-50 | mg |
| rosiglitazon | 5-50 | mg |
| insulin, i.v. | 4-500 | NE/ml |

28. Method of combined therapy treatment according to any of claims 22 to 24 characterised by administering a combination and/or composition according to following effective daily or per hour doses using two active substances:
a) an enzymatic NO donor of the group:

| | | |
|---|---|---|
| NTG retard per os | 0.26-31.2 | mg |
| NTG transdermal | 0.02-0.8 | mg/hour |
| ISDN per os | 0.1-1 | mg |
| ISDN retard per os | 0.2-2 | mg |
| ISDN transdermal | 3-180 | mg |
| ISMN per os | 0.1-1 | mg |
| ISMN retard per os | 0.2-3 | mg |
| ISMN transdermal | 3-300 | mg |

b) and a per os antidiabetic of the group:

| | | |
|---|---|---|
| glibenclamide, per os | 0.75-14 | mg |
| metformin, per os | 50-3000 | mg |
| glyburide | 0.1-100 | mg |
| idazon, per os | 20-600 | mg |
| troglitazon | 20-600 | mg |
| pioglitazon | 5-50 | mg |
| rosiglitazon | 5-50 | mg |
| insulin, i.v. | 4-500 | NE/ml. |

29. Method of combined therapy treatment according to any of claims 22 to 24 characterised by administering a combination and/or composition according to effective daily or per hour doses of claims 22 to 24, using variations of more than two active substances:
enzymatic NO donor organic nitrate, sulphonylurea and biguanide derivative; or
enzymatic NO donor organic nitrate, sulphonylurea and thiazolidinedione derivative; or
enzymatic NO donor organic nitrate, sulphonylurea and insulin; or
enzymatic NO donor organic nitrate, biguanide, and tiadiazolidindione derivative; or
enzymatic NO donor organic nitrate, biguanide derivative and insulin; or
enzymatic NO donor organic nitrate, thiazolidinedione derivative and insulin; or
enzymatic NO donor organic nitrate, sulphonylurea, biguanide and thiazolidinedione derivative; or enzymatic NO donor organic nitrate, sulphonylurea, biguanide, thiazolidinedione derivative and insulin.
